(19) United States
(12) Patent Application Publication
(10) Pub. No.: US 2023/0140486 A1
(43) Pub. Date: May 4, 2023

(54) FUSION PROTEIN AND METHOD OF DETECTING BACTERIA HAVING PSEUDAMINIC ACID

(71) Applicant: Academia Sinica, Taipei (TW)

(72) Inventors: Shih-Hsiung WU, Taipei (TW); I-Ming LEE, Taipei (TW)

(73) Assignee: Academia Sinica, Taipei (TW)

(21) Appl. No.: 17/913,473

(22) PCT Filed: Apr. 14, 2021
(86) PCT No.: PCT/US2021/027292

§ 371 Date: Sep. 22, 2022

Related U.S. Application Data
(60) Provisional application No. 63/011,424, filed on Apr. 17, 2020.

(51) Int. Cl. C07K 14/005; C12Q 1/04

(52) U.S. Cl. CPC C07K 14/005; C12Q 1/04; C07K 2319/60

(57) ABSTRACT

A fusion protein including a phage tail-spike protein ΦAB6TSP and a signal indicator is provided. Also, a method of detecting bacteria having pseudaminic acid (Pse) is provided, including steps of contacting a sample with a phage tail-spike protein ΦDAB6TSP; and detecting a signal from the sample. The fusion protein and the method of detecting bacteria can be applied to a set of practical diagnosis and therapeutic alternative against Pse-coated antibiotic resistant pathogenic bacteria.

### CROSS-REFERENCE TO RELATED APPLICATIONS

The present disclosure claims priority to U.S. Provisional Application Ser. No. 63/011,424 filed on Apr. 17, 2020, the entirety of which is hereby incorporated by reference herein for all purposes.

### FIELD OF THE INVENTION

The present disclosure relates to a fusion protein including a phage tail-spike protein DAB6TSP and a method of detecting bacteria having pseudaminic acid (Pse) using DAB6TSP, which is used to guide the glycol-based antibody with cross-species antimicrobial activity.

### BACKGROUND OF THE INVENTION

Nonulosonic acids are the monosaccharide superfamily that share nine carbon α-keto acid as common core with mostly C-5 and C-7 structural variations leading to over 50 naturally occurring derivatives.¹⁻⁴ Sugars belonging to this family are found to expose on the surface of eukaryotic and prokaryotic organisms, and sialic acid is the most widely distributed and best studied nonulosonate subclass.⁵⁻⁶ See FIG. 1. The subclass unique to bacterial surface glycan or glycoprotein is identified as 5,7-diamino-3,5,7,9-tetradeoxy nonulosonic acids, comprising of two predominant components, peudaminic acid (Pse) and legionaminic acid (Leg)⁷⁻⁸, which are crucial virulence factors led to opportunistic bacteria achieve pathogenicity, but the exact biological function remains intangible.

For Pse, it has been noted that the post-translational Pse decoration is absolutely necessary for functional flagellin assembling in Campylobacter jejuni and Helicobacter pylori to aid motility and colonization. ⁹⁻¹⁰ Pse was also reported on the exopolysaccharide (EPS) of Acinetobacter baumannii and Enterobacter species derived from “ESKAPE” pathogens that is an acronym encompassing six multidrug resistant (MDR) bacteria commonly responsible for the most acute nosocomial infections.¹¹⁻¹⁴ What's worse, the last line antibiotic carbapenem and colistin for management Gram-negative pathogen are getting less effective currently.¹⁵⁻¹⁸ Besides, of particular interest is that Pse may facilitate the bacteria to escape from host immune response due to the structural similarity to eukaryotic sialic acid.¹⁹⁻²⁰ In case of C. jejuni, the binding of Pse on flagella to sialic acid-binding immunoglobulin type lectin 10 (Siglec-10) receptor orchestrated the interleukin 10 (IL-10) expression which mediates the anti-inflammatory effect to suppress natural killer (NK) cell activity.²¹ To date, a Pse isomer, acinetaminic acid (Aci, see FIG. 1), was also found on the A. baumannii EPS and appears to participate in virulence.²²⁻²³ Accordingly, Pse has been greatly addressed an attractive target for clinical diagnosis and therapeutic application over recent years.

Accurate diagnosis is the prerequisite for efficient treatment to combat disease.²⁴ A serious of strategies, such as mass spectrometry, electrochemical sensors and array, have been developed to facilitate diagnosis until now. In fact, the above-mentioned methods are relatively expensive with time-consuming and current advances are trend to quick, reliable and cost-effective.²⁵ Among which, fluorescent-labeled enzymatic probes follow these regards as powerful tools for real-time monitoring of biological events in the living cell.²⁶⁻²⁷ In terms of bacterial surface glycan, using the fluorophore linked to the enzyme that specifically recognizes the critical carbohydrate moiety on glycan renders facile quantification of the glycosylation on the cell surface. Subsequently, the results of probe detection permit glycan-based therapy that aimed at the pathogen-specific carbohydrate, like antibody treatment, as the alternative of antibiotic. Recent advanced clinical trials revealed that several glycol-associated antibodies efficiently kill the bacteria or attenuate the bacterial virulence in infection of Klebsiella pneumoniae and Stapnylococcus aureus.²⁸

In light of the foregoing, Pse is a unique carbohydrate distributed in surface-associated glycan of pathogenic bacteria with the pivotal roles in virulence. Owing to significant antigenicity, Pse was considered as the attractive target for vaccination or antibody-based therapy against acute threat caused by antibiotic resistant bacteria. Along this regard, a probe carrying precise bacterial surface Pse detection is urgently demanding.

### SUMMARY OF THE INVENTION

In order to achieve the aforesaid objective, the present disclosure intends to conjugate the fluorescence with Pse-recognized protein for probing the Pse-coated pathogenic bacteria to guide the glycol-based antibody with cross-species antimicrobial activity.

In an aspect of the present disclosure, a fusion protein including a phage tail-spike protein ΦAB6TSP and a signal indicator is provided.

In another aspect of the present disclosure, a method of detecting bacteria having pseudaminic acid (Pse) is provided, including steps of contacting a sample with a phage tail-spike protein ΦAB6TSP; and detecting a signal from the sample.

Preferably, the bacteria include Acinetobacter baumannii, Helicobacter pylori, Enterobacter cloacae, or Campylobacter jejuni.

Preferably, the method further includes a step of dyeing the sample prior to the contacting step.

Preferably, the method further includes a step of immobilizing the phage tail-spike protein ΦAB6TSP on a substrate.

Preferably, the phage tail-spike protein ΦAB6TSP is fused with a signal indicator.

Preferably, the signal indicator includes luminescent molecule, chemiluminescent molecule, fluorescent dyes, fluorescence quenchers, apatite, colored molecules, radioactive isotope, scintillators, biotin, avidin, streptavidin, protein A, protein G, antibodies or fragments thereof, poly-histidine, Ni²⁺, flag tags, myc tags, HA tags, or enzymes.

Preferably, the signal indicator is DyLight-650.

Preferably, the DyLight-650 is labeled on K261, K307, K432, L476 and/or K547 of the phage tail-spike protein ΦAB6TSP.

Preferably, the phage tail-spike protein ΦAB6TSP includes a catalytic residue mutation.

Preferably, the catalytic residue mutation includes E447Q mutation.

Preferably, the phage tail-spike protein ΦAB6TSP including a sequence having at least 90% identity to SEQ ID NO: 2 or SEQ ID NO: 4.

In the present disclosure, there are at least the following advantages:

1. The present disclosure can be applied as a probe of clinic diagnosis to detect the Pse on the surface of bacteria isolated from the patients.

2. The present disclosure can be applied to guide the anti-Pse therapy to relief the suffer of Pse-coated bacterial infection.

3. The present disclosure can provide simple manipulation by using a ELISA based detection.

4. The present disclosure is cost-effective compared to the conventional methods, such as Mass spectroscopy.

5. The present disclosure endeavored to opened a new avenue on a set of diagnosis and glycol-based therapeutic alternative to the clinical challenge on antibiotic resistance.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1 illustrates structures of commonly occurring nonulosonic acids, including sialic acid (1), pseudaminic acid (2), legionaminic acid (3) and acinetaminic acid (4).

FIG. 2 illustrates (A) fluorescent reagent DyLight-650 labeling mechanism is based on conjugation of NHS ester to the primary amine on the enzyme; (B) D.O.L. of Dy-I-ΦAB6TSP and Dy-serum, respectively; and (C) fluorescent comparison of Dy-I-ΦAB6TSP and Dy-serum treated with different O.D. value of Ab-54149.

FIG. 3 illustrates (A) the complex structure of ΦAB6TSP displayed that K261, K307, K432, K476 and K547 were labeled with Dylight-650, where the residues with asterisk were located on other chain; and (B) a table showing the labeled peptides with corresponded ion scores and molecular weight.

FIGS. 4A-4E illustrate the Mass spectra of labeled peptides of K261, K307, K432, K476 and K547, respectively.

FIG. 5 illustrates respective fluorescent images of bacteria cell membrane stained by CellBrite™ Fix 488 and Dy-I-ΦAB6TSP while Dy-I-ΦAB6TSP incubated with Ab-54149, Ab-SK44, Hp-26695 and Ec-atcc 13047, wherein CellBrite™ Fix 488 showed in green fluorescence and Dylight-650 showed in dark red fluorescence.

FIG. 6 illustrates the merged fluorescence microscopy images of Dy-I-ΦAB6TSP incubated with Ab-54149, Ab-S K44, Hp-26695 and Ec-atcc 13047, respectively.

FIG. 7 illustrates a schematic diagram of cross-species Pse detection using a major Pse detected manner in accordance with the present disclosure.

FIG. 8 illustrates (A) the fluorescence of Ab-54149 binding with Dy-I-ΦAB6TSP compared to other two non Pse-coating A. baumannii strains Ab-SK44 and Ab-SK17R; and (B) Dy-I-ΦAB6TSP demonstrated concentration-dependent to fluorescence upon the consistent O.D. value of Ab-54149.

FIG. 9 illustrates (A) linear regression of the fluorescence of Dy-I-ΦAB6TSP and O.D. value of Ab-54149; (B) linear regression of the fluorescence of Dy-I-ΦAB6TSP and O.D. value of Hp-26695; and (C) linear regression of the fluorescence of Dy-I-ΦAB6TSP and O.D. value of Ec-atcc 13047, wherein data are mean±standard deviation from three independent experiments.

FIG. 10 illustrates the fluorescence of Dy-I-ΦAB6TSP did not show the linear regression to the O.D. values of the non-Pse coated (A) Ab-SK44 and (B)Ab-SK17R.

FIG. 11 illustrates a way of forming PA Hp-26695 by disrupting PseI in Hp-26695 Pse biosynthesis pathway.

FIG. 12 illustrates (A) the fluorescence of Hp-26695 and Hp-11687 binding with Dy-I-ΦAB6TSP compared to PA Hp-2669; (B) linear regression of the fluorescence of Dy-I-ΦAB6TSP and O.D. value of Hp-11687; and (C) the fluorescence of Dy-I-ΦAB6TSP independent to O.D. value of PA Hp-26695.

FIG. 13 illustrates a schematic diagram of cross-species Pse detection using an immobilized Pse detected manner in accordance with the present disclosure.

FIG. 14 illustrates (A) the FITC fluorescence of the I-ΦAB6TSP immobilized plate compared to the glycoconjugate boosted serum immobilized plate; (B) the FITC fluorescence of the same amount of Ab-54149, Ab-SK44 and Ab-SK17R; (C) the FITC fluorescence of Ab-54149 compared to non Pse-coated strains Ab-SK44 and Ab-SK17R on the I-ΦAB6TSP immobilized 96-well plate; and (D) the FITC fluorescence of I-ΦAB6TSP binding FITC fused Ab-54149 in concentration-dependent manner.

FIG. 15 illustrates (A) fluorescence microscopy images of FITC fused Ab-54149, Ab-SK44, Hp-26695 and Ec-atcc 13047 on the I-ΦAB6TSP immobilized 96-well plates, respectively, wherein FITC represented green fluorescence; (B) linear regression of the fluorescence of FITC and O.D. value of Ab-54149; (C) linear regression of the fluorescence of FITC and O.D. value of Hp-26695; (D) linear regression of the fluorescence of FITC and O.D. value of Ec-atcc 13047, where data are mean±standard deviation from three independent experiments.

FIG. 16 illustrates the FITC fluorescence of (A) Ab-SK44 and (B)Ab-SK17R.

FIG. 17 illustrates (A) the fluorescence of FITC fused Hp-26695 and Hp-11687 compared to FITC fused PAHp-26695 on I-ΦAB6TSP immobilized plate; (B) linear regression of the fluorescence of FITC and O.D. value of Hp-11687; and (C) linear regression of the fluorescence of FITC and O.D. value of PA Hp-26695.

FIG. 18 illustrates flow cytometry analysis of binding capacity of glycoconjugate boosted serum toward (A) Hp-26695 and (B) PA Hp-26695.

FIG. 19 illustrates a schematic diagram of cross-species Pse bactericidal activity in accordance with the present disclosure.

FIG. 20 illustrates complement bactericidal assay of glycoconjugate boosted serum toward (A) Hp-26695 and (B) Ec-atcc 13047. The assay was tested with serum in serial dilution (3× to 729×) and percentage of bacterial death is evaluated by counting CFU on each plate (inset). Data are mean±standard deviation from three independent experiments.

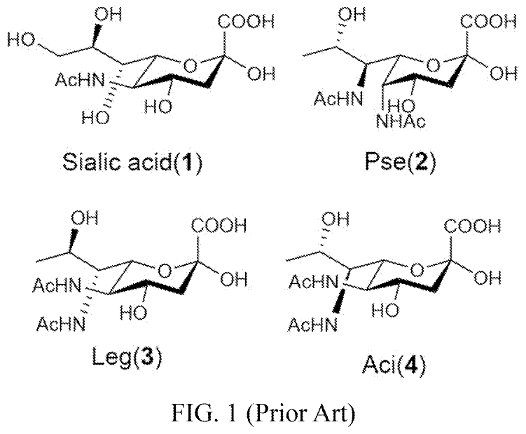

### DETAILED DESCRIPTION OF THE INVENTION

### Definitions

For convenience, certain terms employed in the context of the present disclosure are collected here. Unless defined otherwise, all technical and scientific terms used herein have the same meaning as commonly understood by one of the ordinary skill in the art to which this invention belongs.

The term “fusion protein” as used herein refers to a protein consisting of at least two domains that are encoded by separate genes that have been joined so that they are transcribed and translated as a single unit, producing a single polypeptide.

The term “ΦAB6TSP” as used herein refers to a wild-type protein or a truncated protein.

The term “signal indicator” as used herein can include luminescent molecule, chemiluminescent molecule, fluorescent dyes, fluorescence quenchers, apatite, colored molecules, radioactive isotope, scintillators, biotin, avidin, streptavidin, protein A, protein G, antibodies or fragments thereof, poly-histidine, Ni²⁺, flag tags, myc tags, HA tags, or enzymes.

The term “catalytic residue mutation” as used herein refers to the mutation of amino acid residues which directly involved in the chemistry of catalysis contributing to substrate binding and protein stability.

The term “immobilized/immobilizing” as used herein refers to the act of limiting movement, making incapable movement or retarding movement.

The former studies indicated that a phage ΦDAB6 tail-spike protein (ΦAB6TSP) can digest the exopolysaccharide (EPS) of mild antibiotic resistant Acinetobacter baumannii clinical strain 54149 (Ab-54149), and ΦAB6TSP digested product-CRM197 glycoconjugate boosted serum demonstrated excellent bactericidal activity against Ab-54149.²⁹⁻³⁰ According to the present disclosure, both ΦAB6TSP and the glycoconjugate boosted serum were used to recognize the Pse on Ab-54149 EPS as candidates to be the Pse probe.

### Embodiments

### Experimental Material and Methods

Expression and Purification of I-ΦAB6TSP

The DNA fragments encoding the amino acid sequences of ΦAB6TSP with E447Q mutation were amplified and inserted into the vector pET28a (Novagen) via Ndel and Xhol cloning sites. After the sequences were confirmed, the vectors were transformed into the E. coli BL21(DE3) (Novagen). The cells were grown in Luria-Bertani (LB) medium supplemented with 50 μg/mL kanamycin at 37° C. until the cell density reached OD600 of 0.4-0.6. The cultured cells were induced with 0.1 mM IPTG at 20° C. overnight, and the cells were harvested by centrifugation (6,000 rpm) at 4° C. for 30 min and resuspended in buffer A (25 mM Tris-HCl and 100 mM NaCl, pH 7.5). The cells were lysed by passing through a French Press (Constant System Ltd., Constant System TS 2.2 kw) three times and the lysate was clarified by centrifugation (20,000 rpm) at 4° C. for 60 min. The supernatant was loaded onto an open column filled with nickel-charged chelating resin (Qiagen) and pre-equilibrated with buffer A. The recombinant protein was eluted with 100-300 mM imidazole. The eluted fractions were pooled and then dialyzed against buffer A at 4° C. overnight. The protein was concentrated to ˜10 mg/mL by using a 30K cut-off centrifuge filter (Millipore).

Bacteria Incubated Condition

All A. baumannii strains and E. cloacae ATCC-13047 were growth in Luria Broth under constant aeration at 37° C. and pH near 7.0. For H pylori, the bacteria were growth routinely on Columbia agar base (CAB; Oxoid) containing 10% horse blood and incubated at 37° C. under standard microaerobic atmosphere (5% 02, 10% CO₂, 85% N₂). The single colony was then incubated in BAB medium for overnight under the same condition with shaking speed 200 rpm. Also, the Pse biosynthesis was blocked on Hp-26695 strain via isogenic mutation of pseI gene. The pelI mutant contains chloramphenicol acetyltransferase cassette (CAT) to replace the hp0178 locus in the Hp-26695 genome. The cassette was derived from pAV35 and kindly provided by J. M. Ketley (Department of Genetics, University of Leicester, England)³¹. 1 kbs length of upstream and 600 bps of downstream sequences flanking hp0178 locus were respectively fused with CAT cassette by PCR and then cloned into pGEM7Z. The constructs were delivered to H pylori by natural transformation to generate pelf mutant. The transformants were grown on blood agar plates (BAPs) containing 15 Cm μg ml-1 to select the mutants. The mutant was confirmed by PCR.

Dylight-650 Labeling on I-ΦAB6TSP

Incubating the reagent Dylight-650 and I-ΦAB6TSP followed the molar ratio equal to 1:3 (I-ΦAB6TSP: Dylight-650) in volume 50 μL, for overnight. Next, 200 μL, of dye removal resin (Thermo Fisher Scientific) was loaded into the column and then washed the resin three times with PBS buffer by centrifugation. The labeling protein was loaded into the column and remove unbind dye by centrifugation. Calculating the concentration of labeling protein and degree of labeling were conformed the instruction of Dylight-650 as follows:

(a) Protein concentration (M)=[A₂₈₀−(A₆₅₀×CF)]×diluted factor/_{εprotein}

A₂₈₀=absorbance at 280 nm

A₆₅₀=absorbance at 650 nm

CF=correction factor

diluted factor=the extent (if any) to which the protein:dye sample was diluted for absorbance measurement

_{εprotein}=protein molar extinction coefficient

(b) Degree of labeling (dye molar per mole protein)=A₆₅₀×diluted factor/(_{εfluor}×protein concentration (M))

_{εfluor}=molar extinction coefficient the fluorescent dye

Mass Spectrometry Analysis

Dyligth-650 labeled I-ΦAB6TSP (Dy-I-ΦAB6TSP) and biotin-NHS labeled ΦAB6TSP were treated with 10 mM dithiothreitol (DTT) at 37° C. for 1-2 hours and then added 50 mM iodoacetamide (IAA) at room temperature in dark for 2-4 hours. Afterward, the sample was diluted four times with 25 mM ammonium bicarbonate buffer (pH=8) and incubated with trypsin following the concentration ratio 1 (protein):20 (trypsin) at 37° C. for overnight. Meanwhile, buffer A containing 0.1% trifluoroacetic acid (TFA) with 5% acetonitrile (ACN) and buffer B containing 0.1% TFA with 80% CAN were prepared. 1 mL buffer B is used to activate C-18 tip and then C-18 tip was washed with 1 mL buffer A. After loading the trypsin digested Dy-I-ΦAB6TSP into C-18 tip, the tip was washed with buffer A for several times and then eluted the peptides with buffer B. The sample was lyophilized and further analyzed by Mass.³²

All mass spectrometry in the present disclosure were executed by a LTQFT Ultra (Linear quadrupole ion trap Fourier transform ion cyclotron resonance) mass spectrometer (Thermo Electron, San Jose, Calif.) equipped with a nanoelectrospray ion source (New Objective, Inc.), an Agilent 1100 Series binary high-performance liquid chromatography pump (Agilent Technologies, Palo Alto, Calif.), and a Famous autosampler (LC Packings, San Francisco, Calif.). The digestion solution was injected (6 μl) at 10 μl/min flow rate on to a self-packing precolumn (150 μm I.D.×20 mm, 5 μm, 100 Å). Chromatographic separation was performed on a self-packing reversed-phase C18 nano-column (75 μm I.D.×300 mm, 5 μm, 100 Å) using 0.1% formic acid in water as mobile phase A and 0.1% formic acid in 80% acetonitrile as mobile phase B operated at 300 mL/min flow rate. Survey full-scan MS condition: mass range m/z 200-2000, resolution 50,000 at m/z 400. Electrospray voltage was maintained at 1.8 kV and capillary temperature was set at 200° C.

Fluorescence Measurement with the Major Pse Detected Manner

All bacteria were incubated with 10 μg/mL Dy-I-ΦAB6TSP with gentle shaking for 1 hour. The sample was centrifuged in 4000 rpm for 10 minutes and then discarded the supernatant. The pellet was suspended with PBS subjected to centrifugation (4000 rpm for 10 minutes) for twice as washing steps. Eventually, the pellet was suspended with PBS and transferred to 96 well plate for Dylight-650 fluorescence measurement by Infinite M1000 Pro (Tecan Group Ltd.).

Fluorescent Microscope for Dy-I-ΦAB6TSP and Bacteria Interaction

Labeling of all bacteria followed the above procedure and the Dy-I-ΦAB6TSP labeled pellet was collected. The pellet was suspended with PBS buffer and then stained the bacteria with CellBrite′ Fix Membrane Dye (Biotium) in final concentration of 1× at 37° C. for 15 minutes. To fix the bacteria, 4% paraformaldehyde in 1×PBS was used for 20 minutes at room temperature. 1-2 μL fixed bacteria was loaded on slide glass and mixed with the same volume of mounting solution. After sealing with cover slide, the sample was observed by LSM inverted confocal microscope (Zeiss).

Fluorescence Measurement with the I-ΦAB6TSP Immobilized Plate

5 μg/mL I-ΦAB6TSP was added to the 96-well plate with 100 μL/well at 4° C. for overnight and I-ΦAB6TSP attached on the polystyrene plate via primarily hydrophobic interaction.³³ The solution was removed from the well and washed wells with 3×200 μL/well of PBS buffer. The plate was blocked by adding the 200 μL/well blocking buffer (1×PBS+1% bovine serum albumin) at room temperature for 1 hour. Meanwhile, cultured bacteria (near 10⁹ CFU) were killed by heating at 60° C. for 30 minutes and subsequent to centrifuge to collect the pellet. The pellet was suspended with PBS and added the FITC/DMSO to the suspension with the final concentration 50 μg/mL (the FITC was dissolved in DMSO at 5 mg/mL to prepare the FITC stock and prepared fresh for each experiment). The sample was incubated at room temperature for 2 hours in the dark with gentle agitation. The labeled bacteria were washed twice with centrifugation and suspension and then loaded into the I-ΦAB6TSP immobilized plate with 100 μL/well after removing the blocking buffer. The plate was placed at room temperature for 2 hours and then washed with wells with 3×200 μL/well of PBS buffer. Finally, 100 μL PBS buffer was added to each well for measuring the FITC fluorescence by Infinite M1000 Pro (Tecan Group Ltd.) or observing the well with fluorescent microscope (Zeiss).

Flow Cytometry

Bacteria Hp-26695 and Pse-aberrant Hp-26695 mutant were grown in BAB medium overnight and then diluted to 10⁶ CFU. The diluted bacteria were incubated with 100-diluted (PBS, 1% BSA) glycoconjugate boosted serum for 1 hour in ice. After washing with PBS, the bacteria were incubated with secondary anti-rabbit Alexa Fluor 488-labeled antibodies (Thermo Fisher; 1:400 diluted in PBS with 1% BSA) for 1 hour in ice. After further washing, the bacteria were resuspended in 2 mL PBS and then analyzed by MoFlo XDP flow cytometer (Beckman Coulter). The bacteria incubated with secondary antibodies were only used as negative control.

Serum Bactericidal Assay

Bacteria Hp-26695 and Ec-atcc 13047 were grown in the medium overnight and then diluted to 1:60000 in PBS to approximately 10⁴ CFU/mL. The bacterial sample were distributed into sterile polystyrene 96-well titer plates with 10 μL in each well. The glycoconjugate boosted serum was serially diluted 3-fold (⅓ to 1/729) with PBS, and then 20 μL serum was added into the bacterial suspension for incubation of 15 minutes at 37° C. Serum was heated at 56° C. for 30 minutes to inactivate endogenous complement. After incubation, 30 μL of newborn rabbit complement (BRC) (Bio-Rad) was added to each well, and the samples were incubated at 37° C. for 1.5 hours. Negative controls were comprised of Ab-54149 and BRC only. Each sample and control was tested in triplicate. A 5 μL reaction mixture from each well was spotted onto the LB agar plate or CAB-horse blood plate and subsequently incubated at 37° C. overnight. Resulting CFU were counted on the following day to determine the bactericidal activity. Subtraction CFU of ⅓ diluted serum from CFU of negative control was set to 100% bacterial death and the bacterial death percentage of other diluted serum was evaluated by the same strategy.

Results

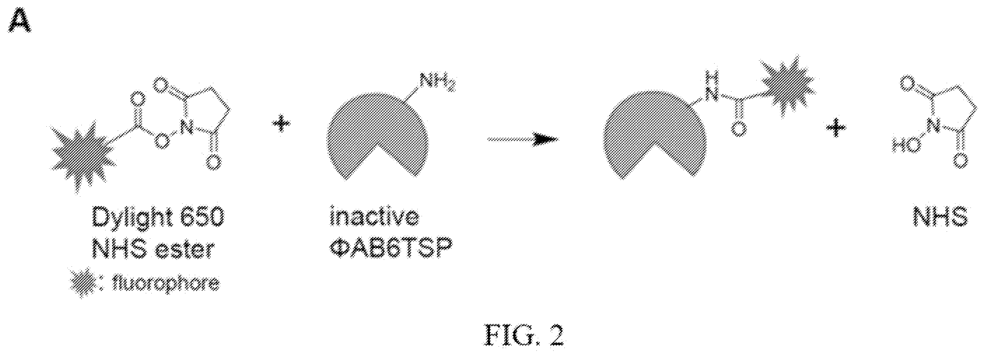

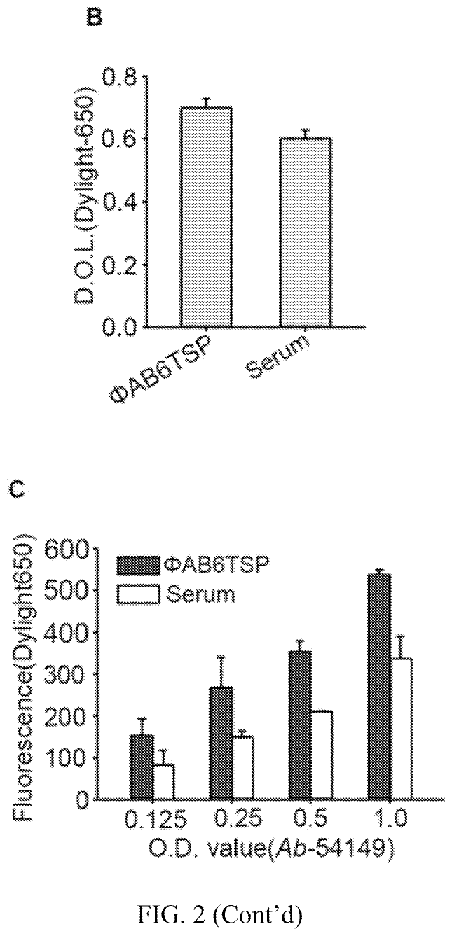

Please refer to FIG. 2. With fluorescent strategy, the present disclosure aimed to connect the catalytic residues-mutated (inactive)ΦAB6TSP (I-ΦAB6TSP) that eliminated the glycohydrolase activity to prolong enzyme on bacteria cell surface or the glycoconjugate boosted serum with the high performance fluorescent dye Dylight-650, of which an N-hydroxysuccinimide (NHS) ester moiety on dye formed a stable amide bond with exposed lysine residues on proteins (FIG. 2, part A). Parts B and C of FIG. 2 illustrate I-ΦAB6TSP was more appropriate to be a Pse probe than glycoconjugate boosted serum under fluorescent strategy. Here, Dylight-650 labeled I-ΦAB6TSP (Dy-I-ΦAB6TSP) had higher degree of labeling (D.O.L) as well as more apparent bacteria O.D. value-dependent fluorescence in Ab-54149 treatment than Dylight-650 labeled glycoconjugate boosted serum (Dy-serum), indicating that I-ΦAB6TSP was more appropriate to be a fluorescent Pse probe than glycoconjugate boosted serum.

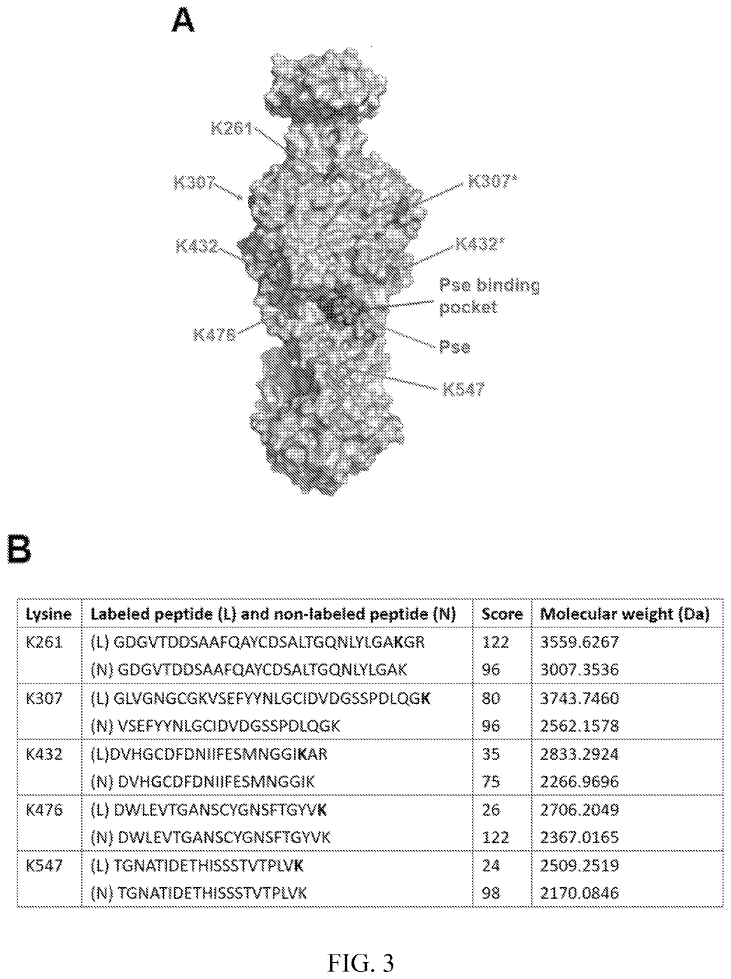

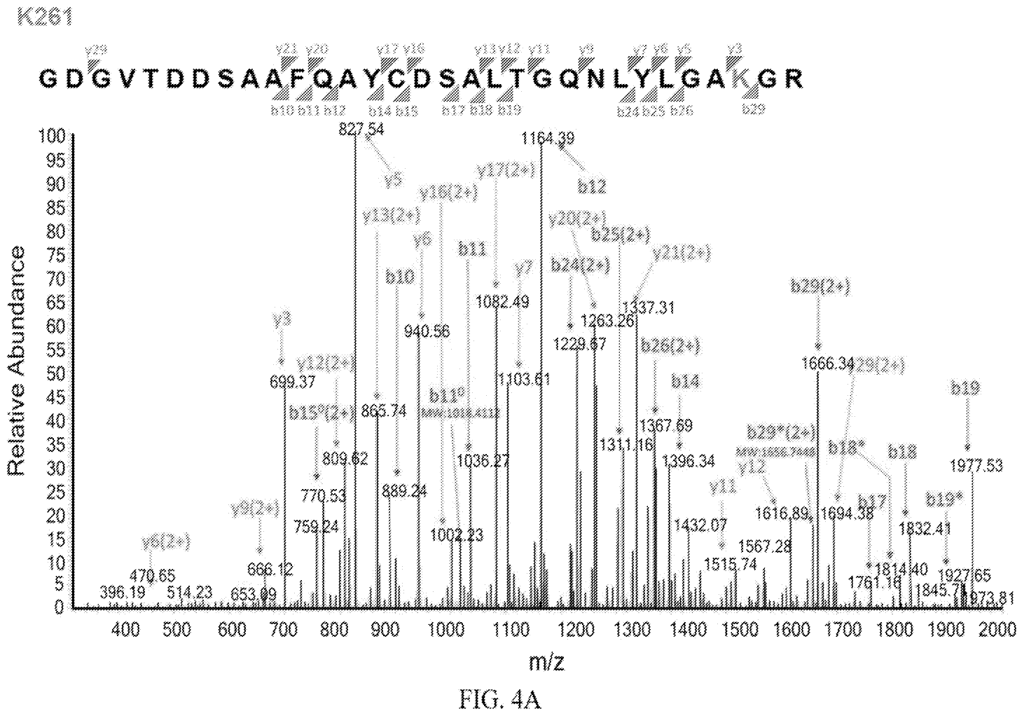

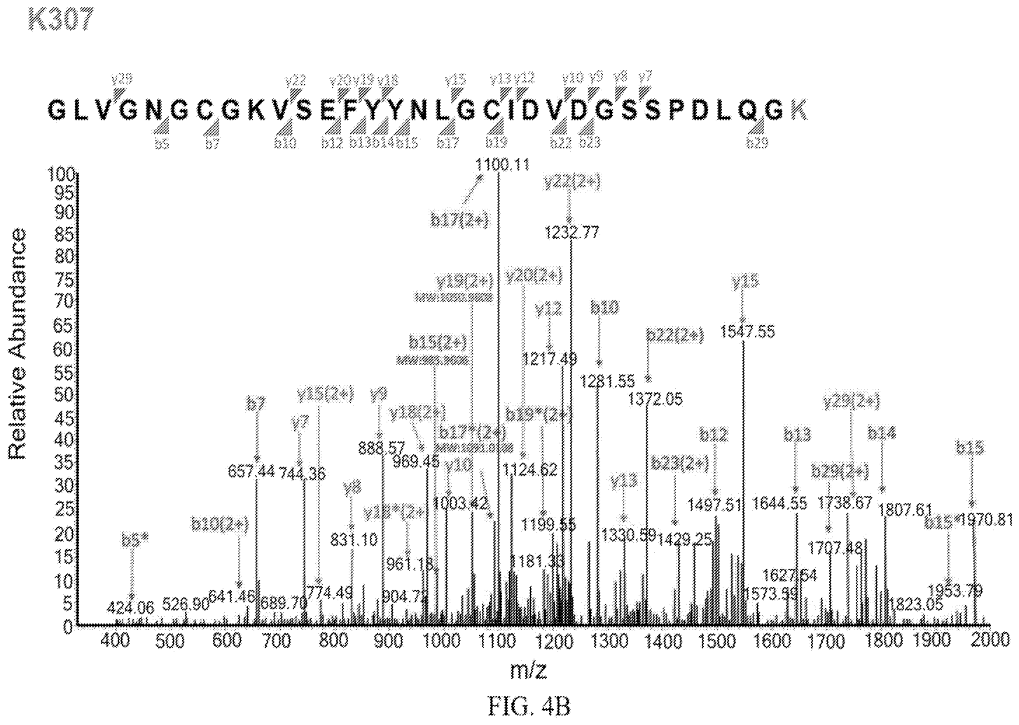

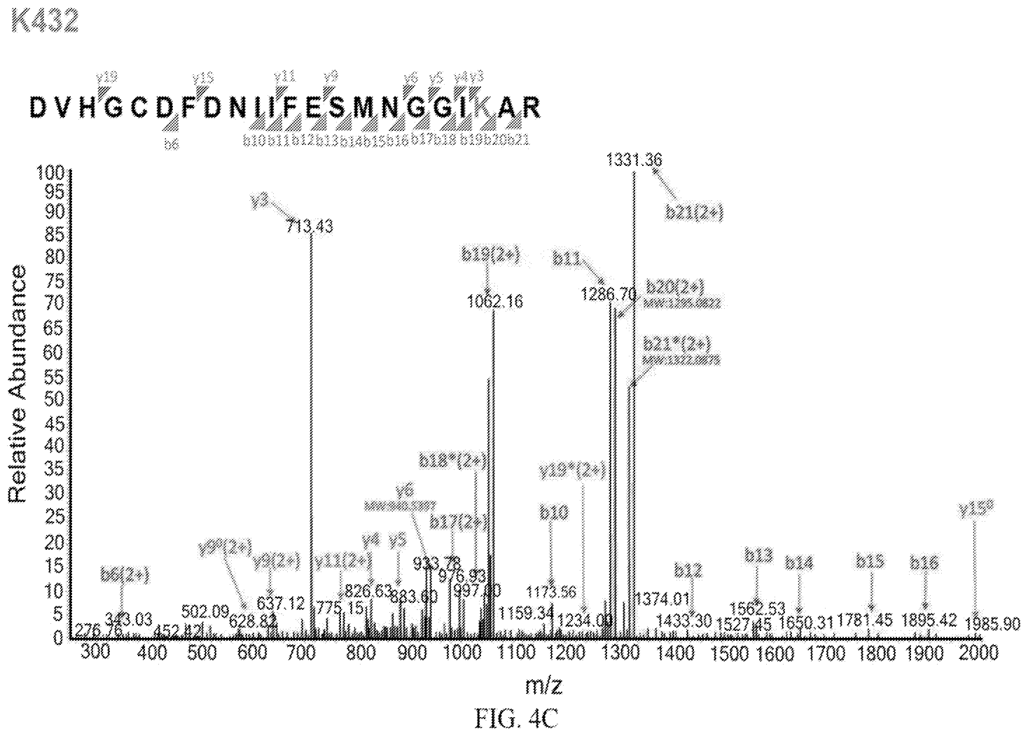

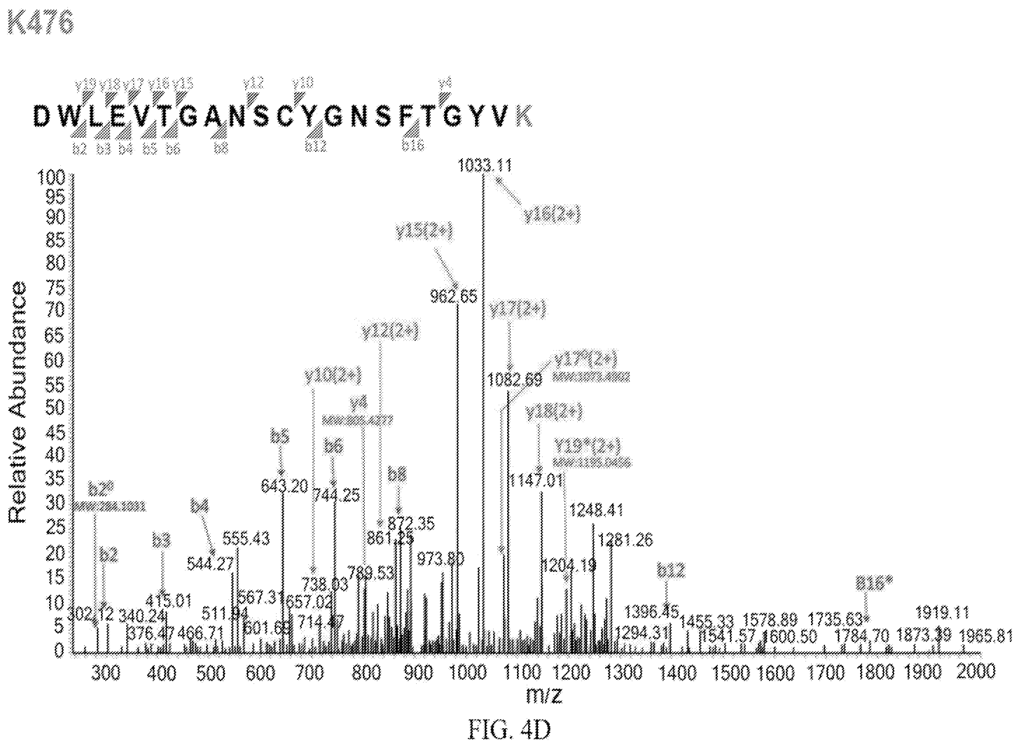

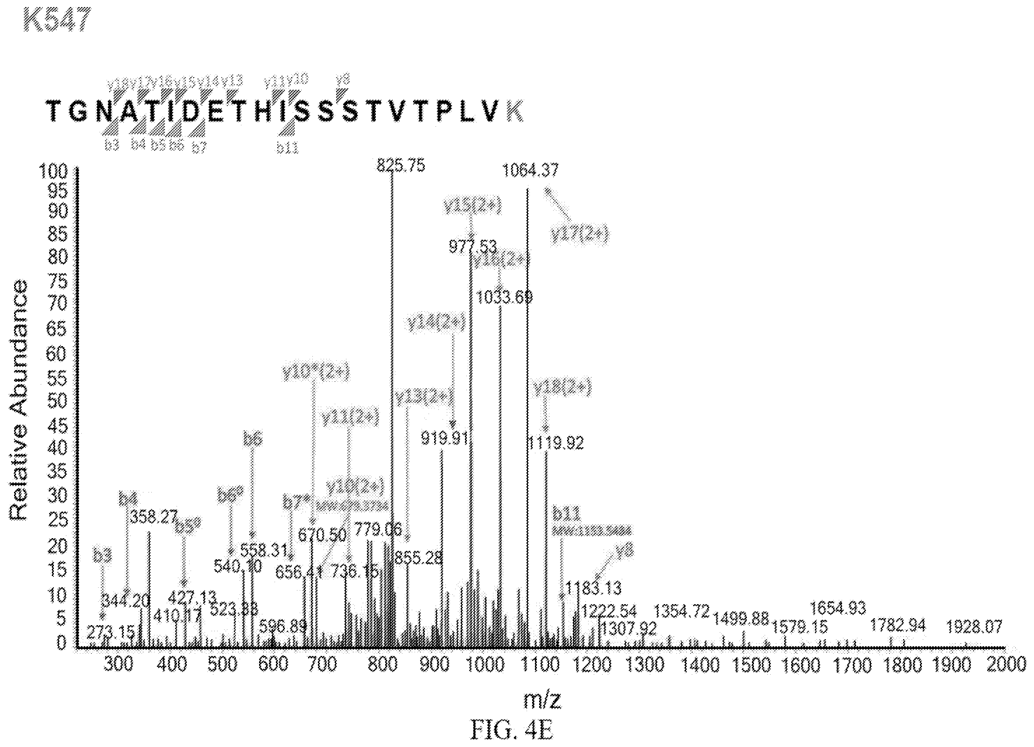

Please refer to FIGS. 3 and 4A-4E. To identify Dylight-650 labeling sites on I-ΦAB6TSP, biotin-NHS reagent treated I-ΦAB6TSP were analyzed by Mass. Mass spectrum revealed that lysine K261, K307, K432, K476 and K547 were labeled with NHS reagent, inferring that Dylight-650 labeling did not spatially interrupt the Pse binding of I-ΦAB6TSP.

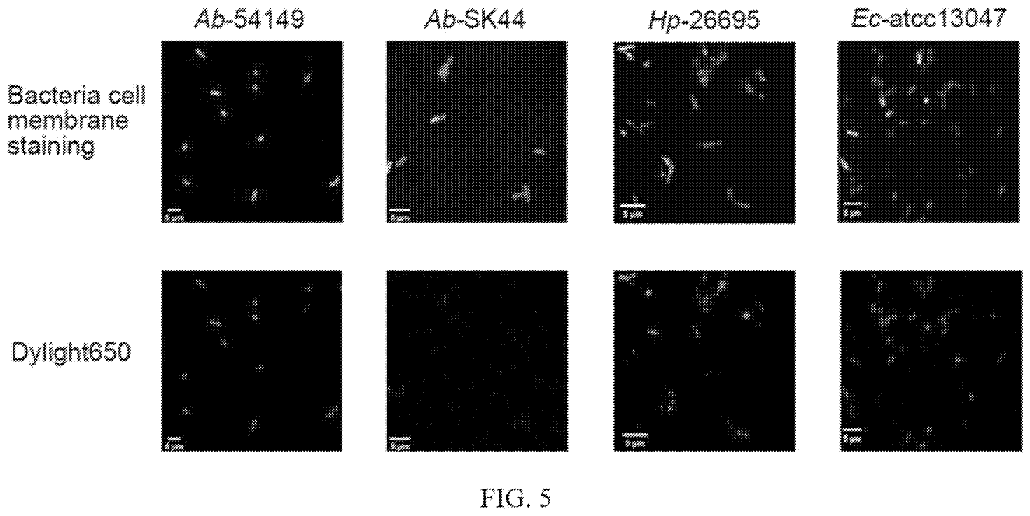

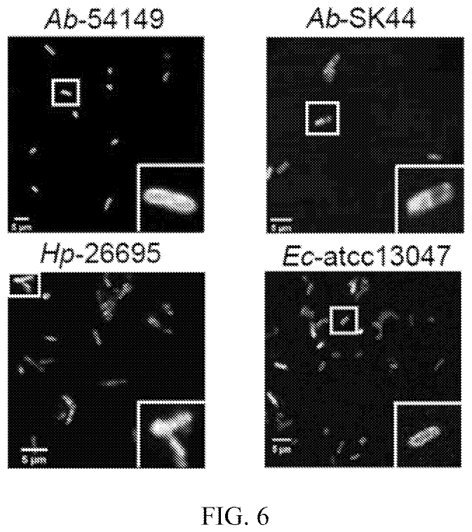

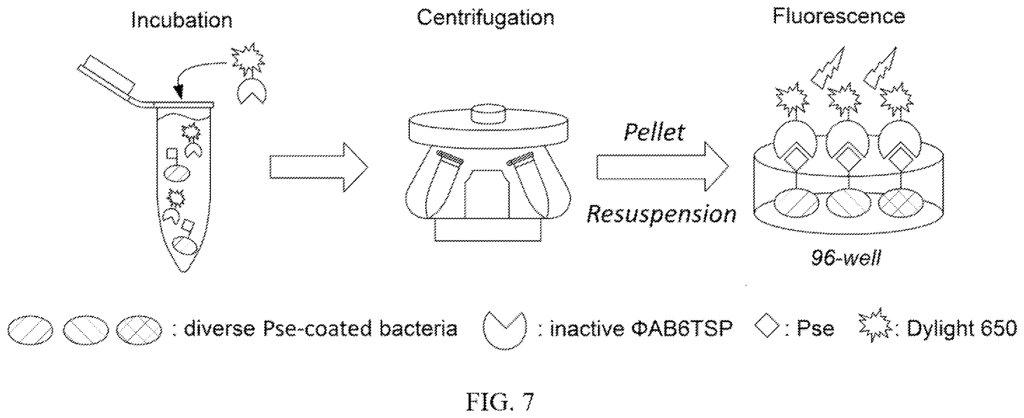

FIGS. 5 and 6 illustrate that Dy-I-ΦAB6TSP can detect cross-species Pse-coated pathogenic bacteria. As shown in figures, bacteria cell membrane was stained by CellBrite™ Fix 488 with green fluorescence and DyLight-650 represented dark red fluorescence. Merging two colors showed near white color (FIG. 6). Indeed, Dy-I-ΦAB6TSP was actually situated on the surface of Ab-54149 compared to no Pse-coated strain Ab-SK44 under the fluorescent microscope. Hence, Dy-I-ΦAB6TSP was reacted with suspended bacteria and then centrifuge to discard the unbound Dy-I-ΦAB6TSP in the supernatant and measure the fluorescence of Dy-I-ΦAB6TSP on the pellet as a major Pse detected manner (FIG. 7).

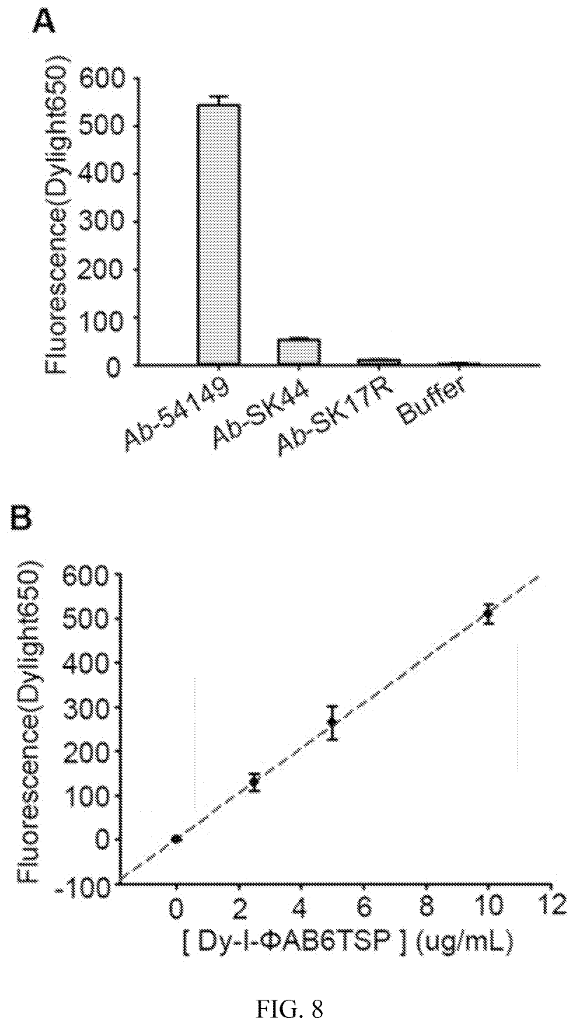

Dy-I-ΦAB6TSP can bind Ab-54149 in concentration-dependent manner. As shown in part A of FIG. 8, Ab-54149 displayed the significant binding with Dy-I-ΦAB6TSP by fluorescence of Dylight-650 compared to the other two A. baumannii strains (Ab-SK44 and Ab-SK17R) without Pse on cell surface. Refer to part B of FIG. 8, raising fluorescence of Dylight-650 conformed with the concentration of Dy-I-ΦAB6TSP upon the consistent O.D. value of Ab-54149, implying that the strategy of Dy-I-ΦAB6TSP for Pse detection is feasible. Inserted pictures showed the pellets after Dy-I-ΦAB6TSP and bacteria incubation. It can be observed a cyan color on the pellet which were from Dylight-650 reagent. Notably, the cyan color from Dy-I-ΦAB6TSP on Ab-54149 pellet that aids to simply judge the existence of Pse.

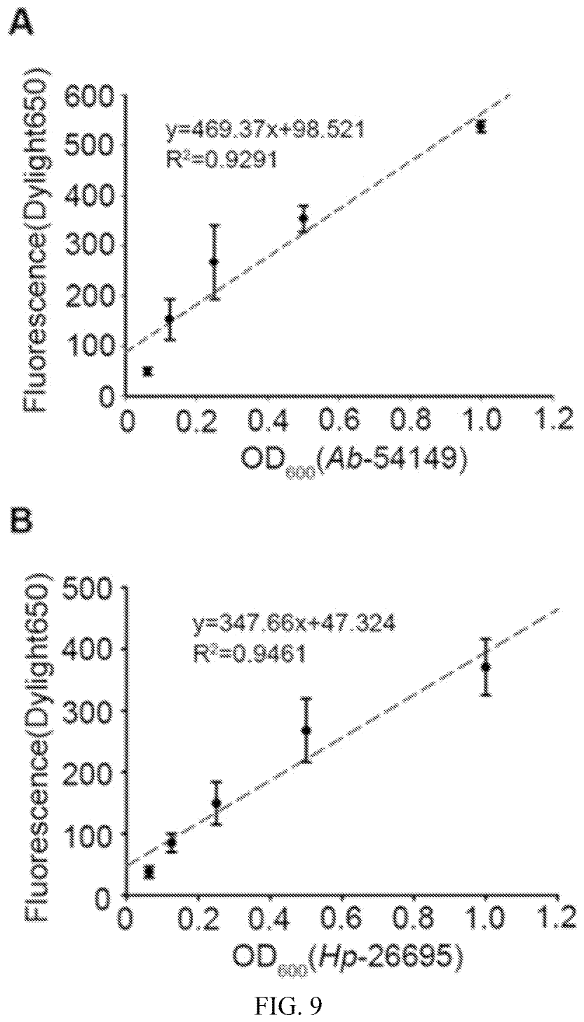

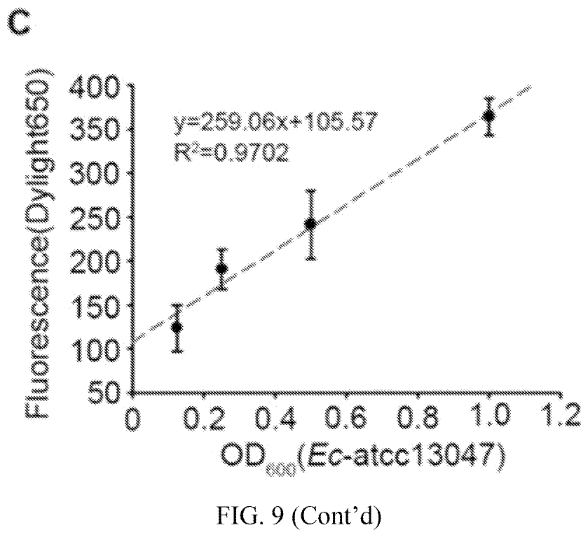

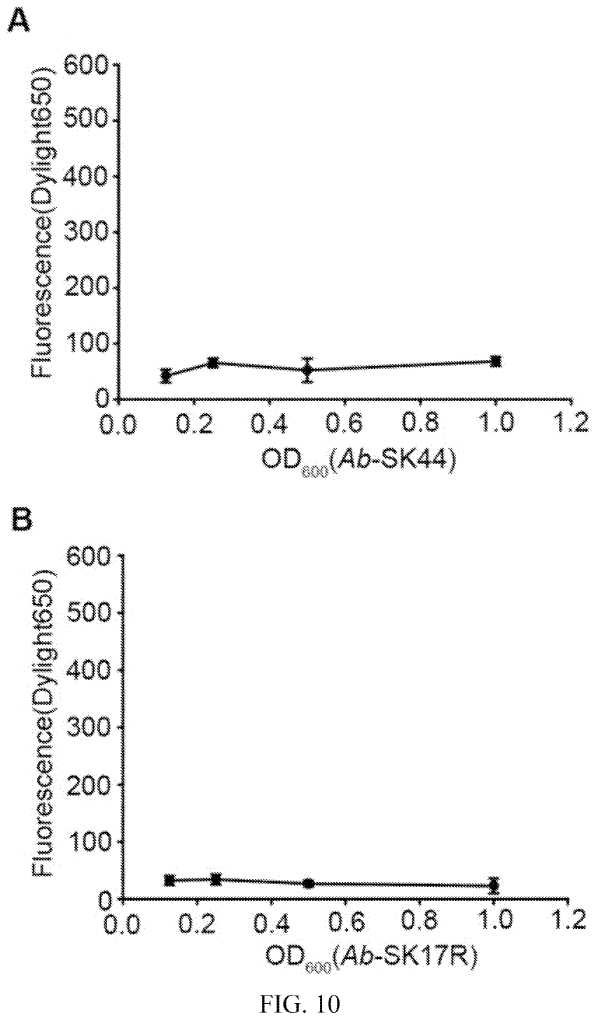

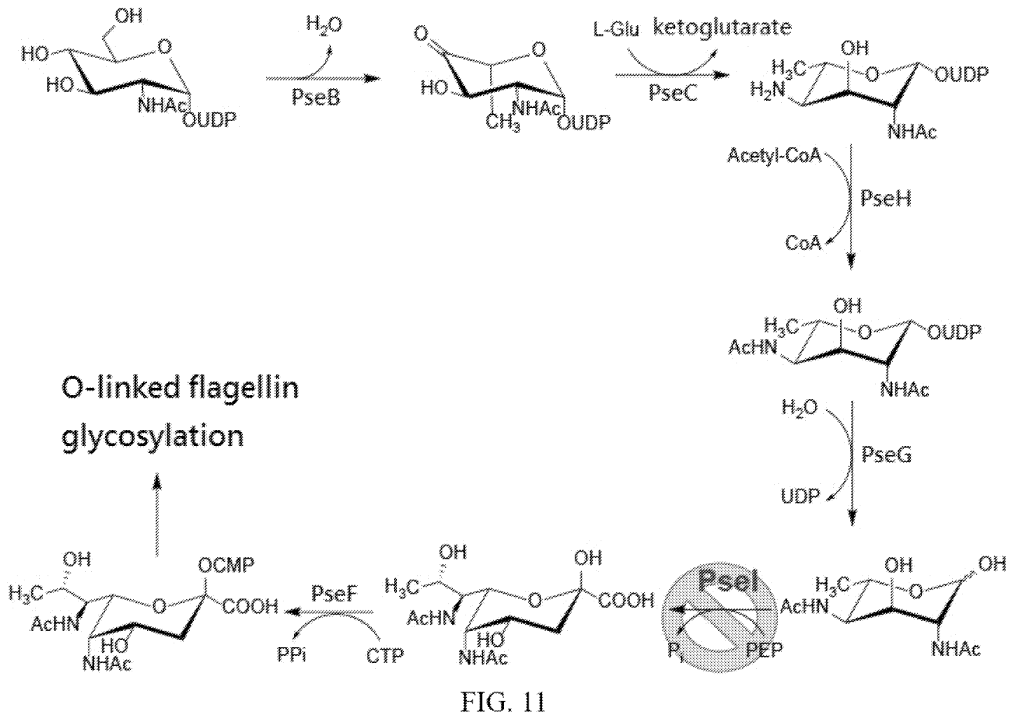

Referring to FIG. 9, the fluorescence of Dy-I-ΦAB6TSP and O.D. value of Ab-54149, Hp-26695 and Ec-atcc 13047 were measured and calculated with linear regressions, respectively. The fluorescence of Dy-I-ΦAB6TSP virtually turned into near proportional to the O.D. value of Ab-54149 that benefit quantification of bacterial surface Pse (FIG. 9, part A). By contrast, the fluorescence was irrelevant to the O.D. value of non-Pse coated Ab-SK44 and Ab-SK17R (FIG. 10, parts A and B).

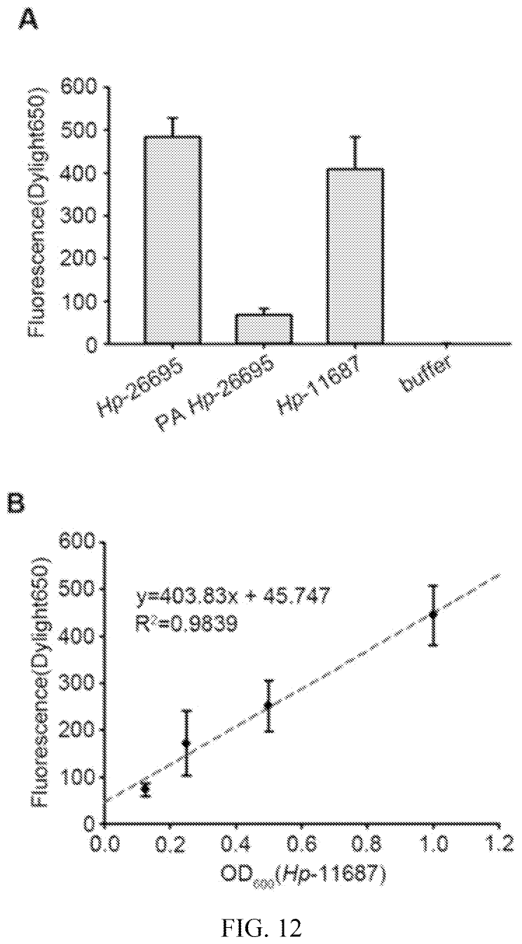

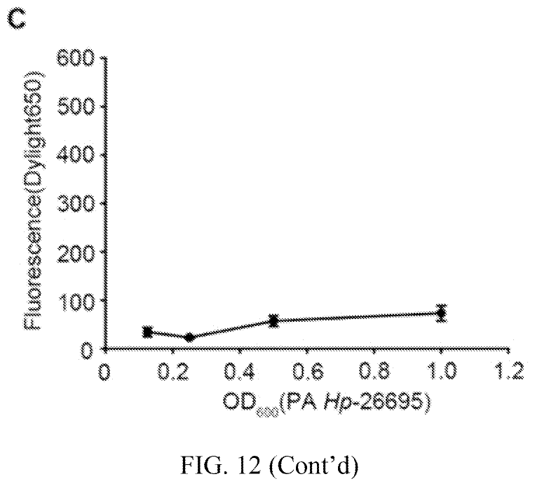

Besides A. baumannii, it was inspected the capacity of Dy-I-ΦAB6TSP to track other remarkable Pse-coated pathogenic bacteria, including well-known Helicobacter pylori strains (Hp-26695 and Hp-11687) and Enterobacter cloacae strain atcc-13047 (Ec-atcc 13047) that was suggested to possess a broad range of antibiotic resistance.³⁴⁻³⁵ Fluorescent microscope revealed that Dy-I-ΦAB6TSP was also found on the surface of Hp-26695 and Ec-atcc 13047 (FIGS. 5 and 6). Referring to FIG. 11, the pseudaminic acid synthase (PseI) in Hp-26695 Pse biosynthesis pathway was disrupted to form Pse-aberrant Hp-26695 mutant (PAHp-26695) as comparison group.³⁶ As shown in FIG. 12, Hp-26695 and Hp-11687 represented the manifest fluorescence of Dy-I-ΦAB6TSP in contrast with PA Hp-26695. Thus, Hp-26695 and Hp-11687 showed significant binding with Dy-I-ΦAB6TSP compared to PA Hp-26695 when same amount of three H pylori strains were treated following the major detected manner. As expected, Hp-26695, Hp-11687 and Ec-atcc 13047 exhibited similar fluorescent tendency to Ab-54149 when executing a major Pse detected manner but not PAHp-26695, verifying that I-ΦAB6TSP was an ideal probe for cross-species bacterial Pse detection (FIGS. 9 and 12).

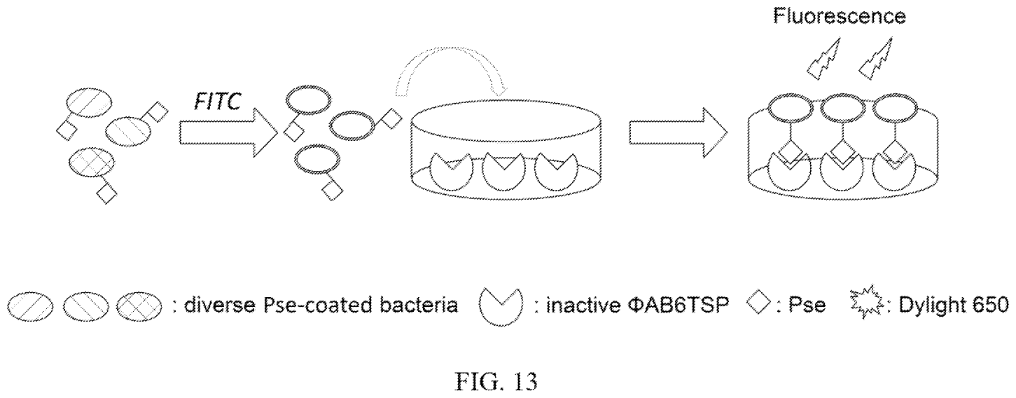

However, the major Pse detected manner was not proper to the bacteria that remained suspended form after centrifugation. In such cases, an immobilized Pse detected manner is adopted by binding FITC fused Ab-54149 on the I-ΦAB6TSP immobilized 96-well plate. Referring to FIG. 13, the fluorescein isothiocyanate (FITC) fused bacteria was incubated in the 96-well plate immobilized with Pse-recognized proteins and measured the fluorescence of FITC after buffer-washing.

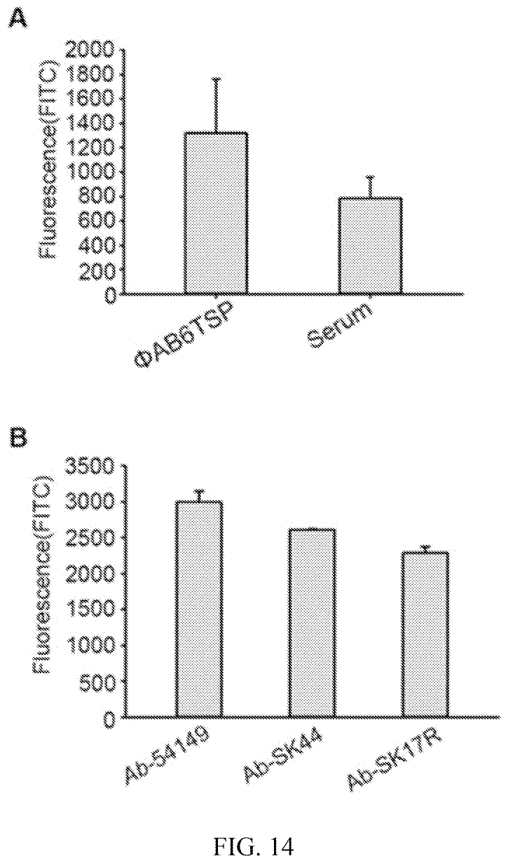

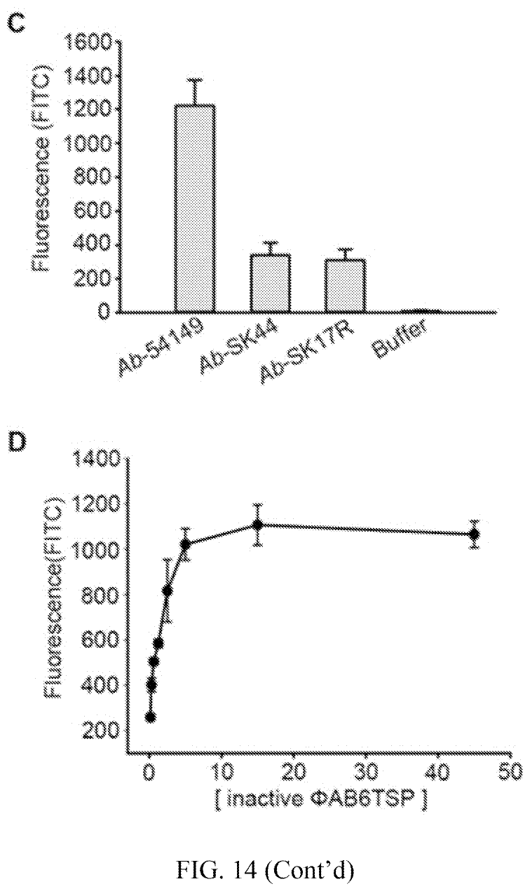

Please further refer to FIG. 14. with the same O.D. value of Ab-54149, the I-ΦAB6TSP immobilized plate can bind more FITC fused Ab-54149, resulting in higher FITC intensity than the glycoconjugate boosted serum immobilized plate. That is, with the same amount of FITC fusedAb-54149, the I-ΦAB6TSP immobilized plate could retain more bacteria that allowed higher FITC fluorescent intensity than the glycoconjugate boosted serum immobilized plate; hence, the I-ΦAB6TSP immobilized plate was selected for further experiments (FIG. 14, part A). On the trial of above A. baumannii strains, all strains possessed similar FITC fused efficiency because same amount of bacteria, Ab-54149, Ab-SK44 and Ab-SK17R, showed similar fluorescent intensity (FIG. 14, part B). Referring to part C of FIG. 14, on the I-ΦAB6TSP immobilized 96-well plate, Ab-54149 showed higher FITC fluorescent intensity than non Pse-coated strains Ab-SK44 and Ab-SK17R. Further referring to part D of FIG. 14, I-ΦAB6TSP can bind FITC fused Ab-54149 in concentration-dependent manner and FITC fluorescence was saturated when the concentration of I-ΦAB6TSP coated on 96-well plates was over 5 μg/mL. Therefore, resembling the results of major Pse detected manner, FITC fluorescence on the Ab-54149 was more immense than that of Ab-Sk44 and Ab-Sk17 and positive correlated to the concentration of I-ΦAB6TSP that was lower than 5 μg/mL.

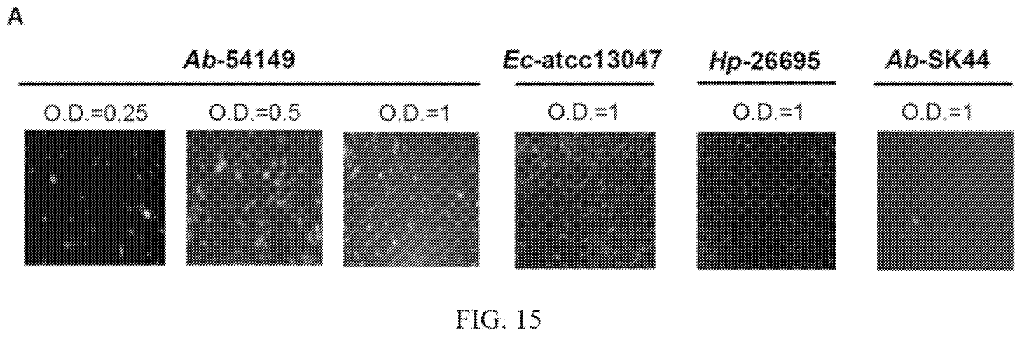

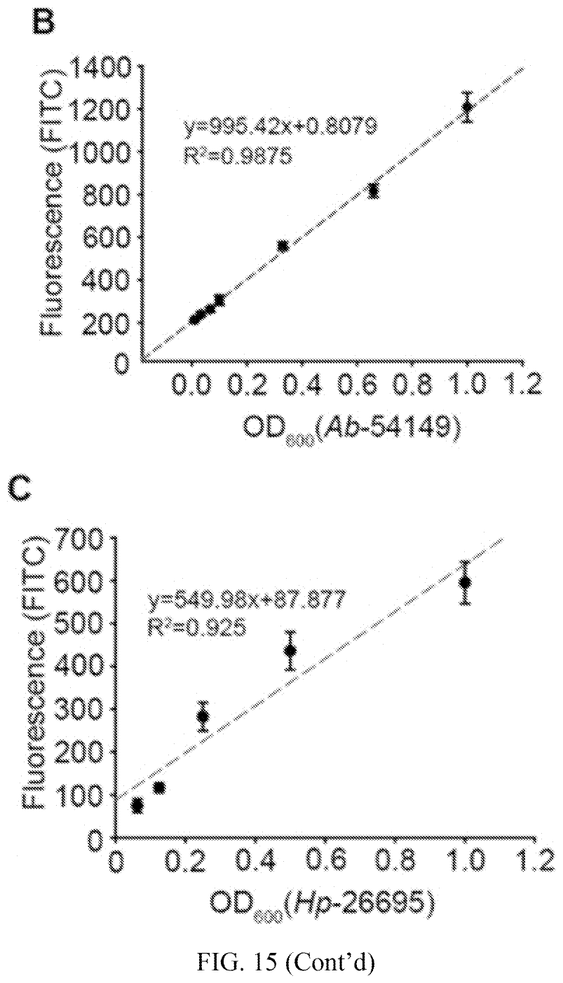

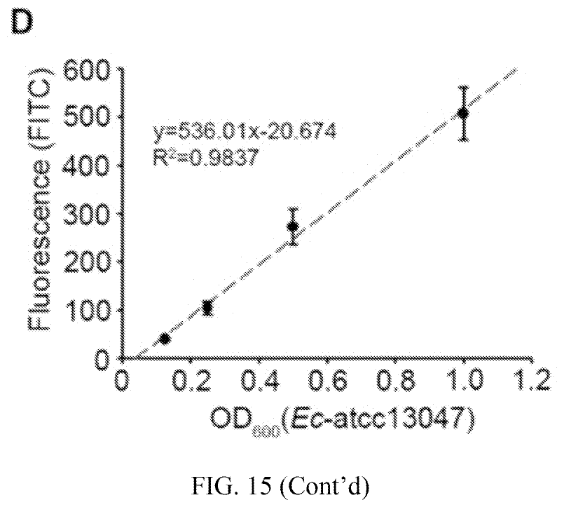

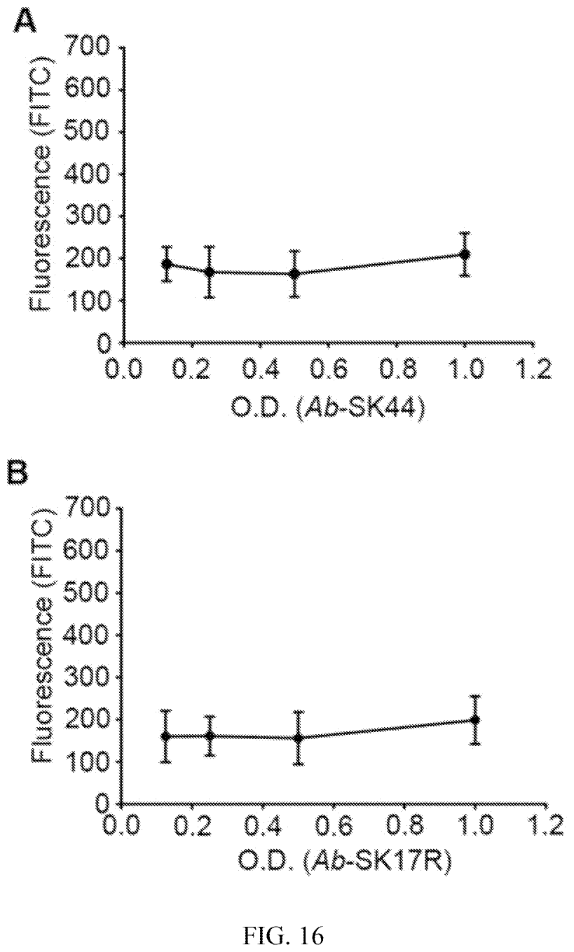

Please refer to FIGS. 15 and 16. FIG. 15 illustrates diverse Pse-coated pathogenic bacteria with FITC fusion can be bound on the I-ΦAB6TSP immobilized 96-well plates. However, the data of FIG. 14 were corresponded to the FITC fluorescent images that displayed more fluorescence upon more O.D. value of Ab-54149 but fluorescence was diminished when Ab-Sk44 was used for test (FIG. 15, part A). Likewise, referring to part B of FIG. 15 and FIG. 16, the fluorescence of FITC was also near proportional to the O.D. value of Ab-54149 contrary to Ab-SK44 and Ab-SK17R by following this procedure. The fluorescence of FITC did not show the linear regression to the O.D. value of the non-Pse coated Ab-SK44 and Ab-SK17R.

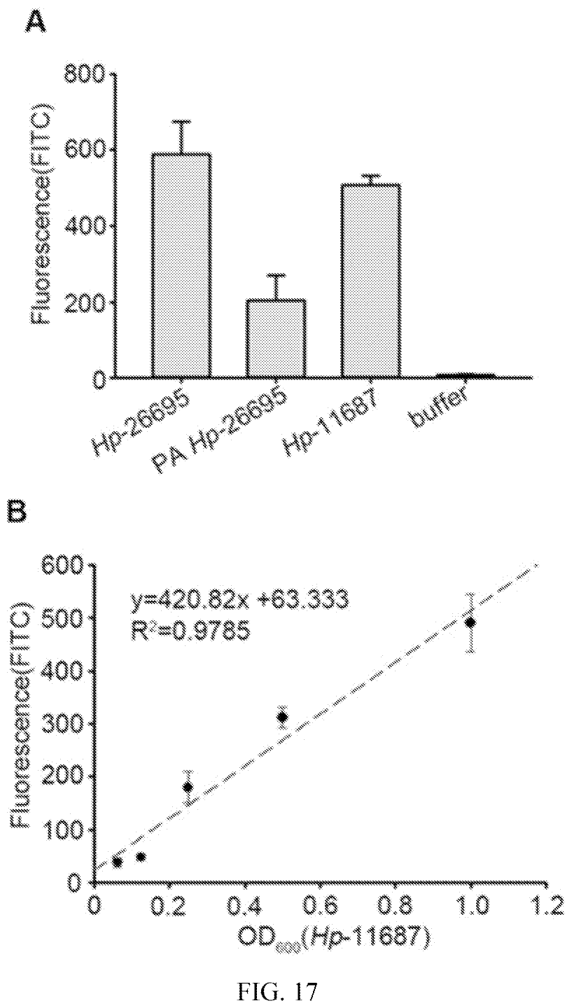

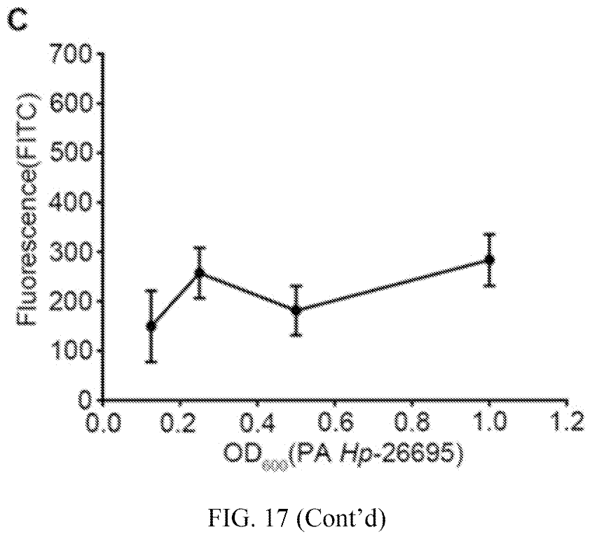

Further referring to FIG. 17, the I-ΦAB6TSP immobilized plate can bind FITC fused Hp-26695 and Hp-11687 but not the FITC fused PA Hp-26695. The fluorescent intensity of FITC fused Hp-26695 and Hp-11687 was more significant than FITC fused PA Hp-26695 on I-ΦAB6TSP immobilized plate when the same amount of three H pylori strains were treated following the I-ΦAB6TSP immobilized procedure. Also refer to FIG. 15 together. With respect to cross-species test, the fluorescent microscope images illustrated that the I-ΦAB6TSP immobilized plate can keep Hp-26695 in agreement with its obvious FITC fluorescence. FITC fluorescence of Hp-11687 on the plate can be found as well but not PA Hp-26695. The FITC fluorescence of FITC did not show the linear regression to the O.D. value of PA Hp-26695. The O.D. value of Hp-26695, Hp-11687 and Ec-atcc 13047 and respective FITC fluorescence were linear regression whereas the O.D. value of PA Hp-26695 was independent to the fluorescence. Accordingly, probing bacterial Pse with I-ΦAB6TSP immobilized plate illustrated similar tendency to the major Pse detected manner but required more manipulated steps and time.

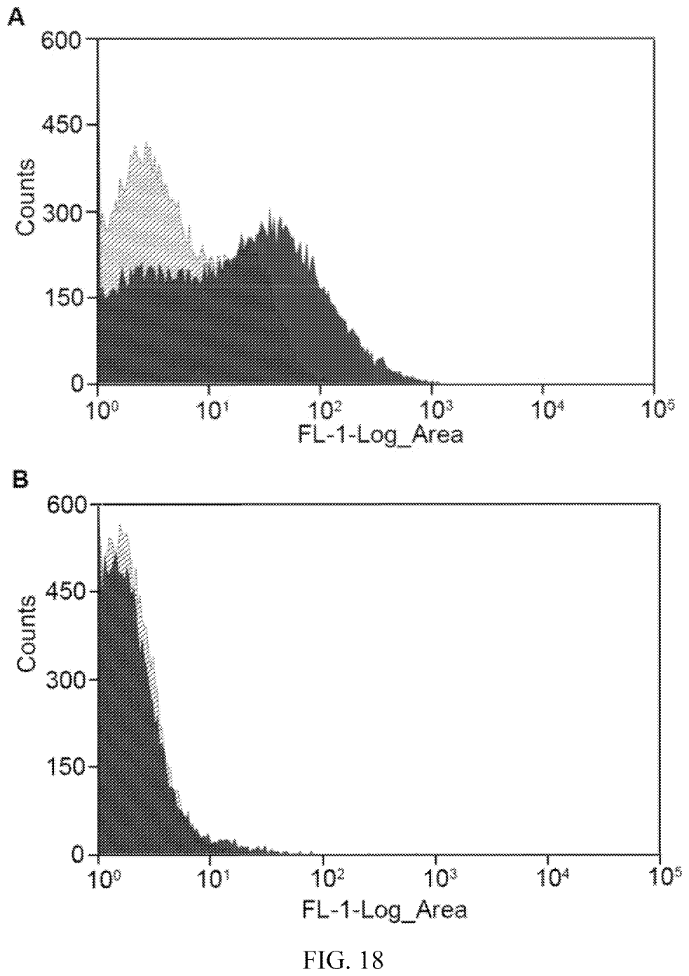

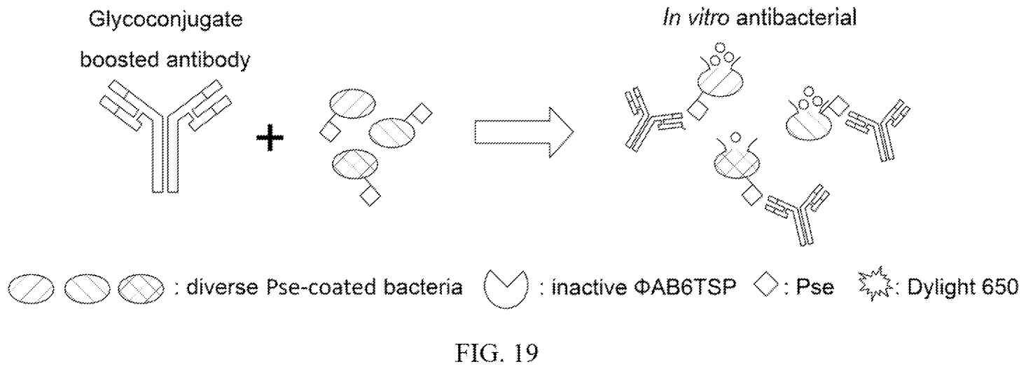

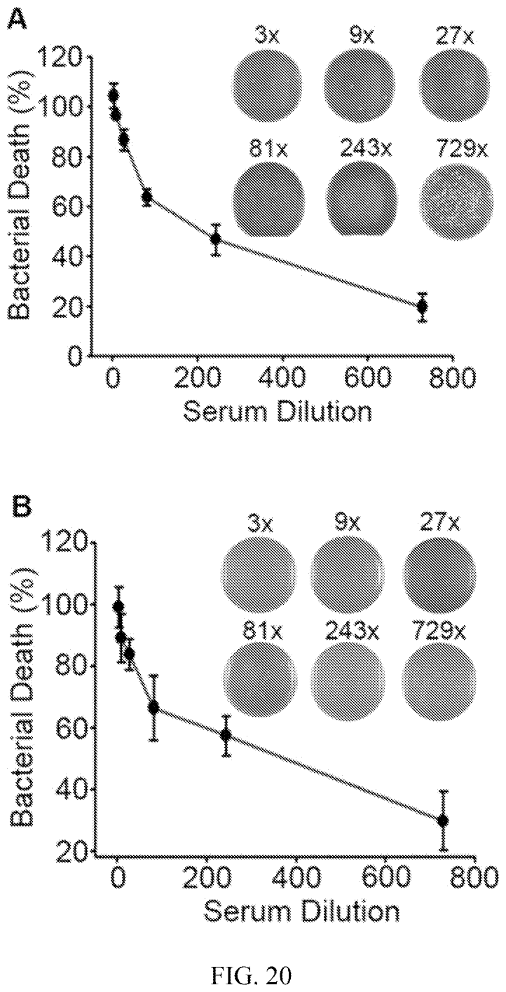

The former study also confirmed that the glycoconjugate boosted serum demonstrated strong antigenicity toward Pse on Ab-54149 EPS and conducted the bactericidal activity. These results intimated that glycoconjugate boosted serum enable to work on the Pse decoration on H pylori and E. cloacae. Referring to FIG. 18, fluorescent signals obtained with positive (bacteria incubated with boosted serum and fluorescently labeled secondary antibodies) and negative (bacteria incubated with fluorescently labeled secondary antibodies only) controls were colored in green and gray, respectively. The significant difference of the fluorescent strength between positive and negative controls indicated that antibodies in the boosted serum definitely bound on the surface of Hp-26695 but not the PA Hp-26695. That is, the glycoconjugate boosted serum specifically bound on flagella of Hp-26695 rather than PA Hp-26695 by flow cytometry analysis, indicating that the glycoconjugate boosted serum can recognize the Pse on not only A. baumannii but also other Pse-coated pathogenic bacteria. Intriguingly, the glycoconjugate boosted serum remarkably killed alive Hp-26695 and Ec-atcc 13047 in vitro through complement bactericidal assay, arising in a potent therapeutic alternative with cross-species activity. ((FIGS. 19 and 20).

Here, as stated above, the catalytic residues-mutated (inactive) ΦAB6TSP (I-ΦAB6TSP) was found to maintain the binding ability toward Pse and represented better labeled and detected efficiency with fluorescent reagent DyLight-650 than glycoconjugate boosted serum. Therefore, the present disclosure utilized DyLight-650 labeled inactive ΦAB6TSP (Dy-I-ΦAB6TSP) to incubate with suspended bacteria and subsequently quantify the fluorescence of pellet as major detected manner. Notably, the fluorescence of DyLight-650 on pellet was near proportional to the OD value of Ab-54149 as well as other Pse-coated pathogenic bacteria like Helicobacter pylori and Enterobacter cloacae, revealing that I-ΦAB6TSP is a promising probe for detecting cross-species Pse. For the few bacteria that do not form pellet after centrifugation, the present disclosure measured the fluorescence of fluorescein isothiocyanate (FITC) fused bacteria in the I-ΦAB6TSP immobilized plate and displayed the similar results to major manner but it required more steps and time. Furthermore, the glycoconjugate boosted serum also exhibited the sensitivity to Pse on H pylori and enable to kill alive H. pylori and E. cloacae, indicating the high potential against cross-species antibiotic resistant bacterial infection.

### CONCLUSION

Over the decade, the pathological roles of nonulosonic acids have drawn attention to development of clinical diagnosis and novel therapy against antibiotic resistant bacteria infection, especially for Pse. Based on the trait of Pse recognition, the fluorescent labeled I-ΦAB6TSP can initially probe surface Pse on bacteria with the color of pellet and the intensity of fluorescence. On cross-species test, the respective spatial configuration of surface glycan on diverse bacteria modulated I-ΦAB6TSP and Pse binding, resulting in the relative fluorescence under same O.D. value that may hinder the judgement. Even so, the linear regression between fluorescence and O.D. value in range of 0.125 to 1 indicated the compelling cross-species Pse detection to be a high potent probe in diagnosis. Using fluorescent-lectin conjugated derivatives for monitoring sugar moiety has been more robust than metabolic labeling and chemoselective ligation that encountered some drawbacks mainly due to the rare recognition of biosynthesis enzymes on various non-native substrates, weak labeling efficiency and high toxicity of catalyst. And definitely, this strategy is cost-effective and simple manipulation. Furthermore, the serum boosted by the glycoconjugate derived from Pse-coated A. baumannii EPS enable to bind the Pse on other pathogenic bacteria, leading to cross-species antimicrobial activity. This consequence suggested the multivalency of Pse-derived glycoconjugate boosted serum against bacteria that may relieve the challenge of antibiotic resistance. To summarize, these efforts become the bellwether on a set of practical diagnosis and therapeutic alternative against Pse-coated antibiotic resistant pathogenic bacteria.

### REFERENCES

1. Tomek, M. B., Janesch, B., Maresch, D., Windwarder, M., Altmann, F., Messner, P., Schaffer, C. Glycobiology 2017, 27 (6), 555.
2. Lewis, A. L., Lubin, J. B., Argade, S., Naidu, N., Choudhury, B., Boyd, E. F. Appl. Environ. Microbiol. 2011, 77 (16), 5782.
3. Bloch, S., Tomek, M. B., Friedrich, V., Messner, P., Schäffer, C., Interface Focus 2019, 9 (2)
4. Kandiba, L., Eichler, J. FEMS Microbiol. Lett. 2013, 345 (2), 110.
5. Lewis, A. L., Desa, N., Hansen, E. E., Knirel, Y. A., Gordan, J. I., Gagneux, P., Nizet, V., Varki, A. Proc. Natl. Acad. Sci. US.A 2009, 106, 13552.
6. Chen, X., Varki, A. ACS Chem. Biol. 2010, 5, 163.
7. Matthies, S., Stallforth, P., Seeberger, P. H., J. Am. Chem. Soc. 2015, 137 (8), 2848.
8. Carter, J. R., Kiefel, M. J., RSC Advances 2018, 8 (62), 35768.
9. Salah Ud-Din, A. I. M., Roujeinikova, A., Cell Mol. Life Sci. 2018, 75 (7), 1163.
10. Hitchen, P., Brzostek, J., Panico, M., Butler, J. A., Morris, H. R., Dell, A., Linton, D., Microbiology 2010, 156 (Pt 7), 1953.
11. Vila, J., Pachon, J. Expert Opin. Pharmacother. 2012, 13, 2319.
12. Kenyon, J. J., Marzaioli, A. M., Hall, R. M., De Castro, C. Glycobiology 2014, 24 (6), 554.
13. Davin-Regli, A., Pages, J. M. Front Microbiol. 2015, 6, 392.
14. Perepelov, A. V., Wang, M., Filatov, A. V., Guo, X., Shashkov, A. S., Wang, L., Knirel, Y. A. Carbohydr Res. 2015, 407, 59.
15. Gonzalez-Villoria, A. M., Valverde-Garduno, V. J. Pathog. 2016, 2016, 7318075.
16. Qureshi, Z. A., Hittle, L. E., O'Hara, J. A., Rivera, J. I., Syed, A., Shields, R. K., Pasculle, A. W., Ernst, R. K., Doi, Y Clin. Infect. Dis. 2015, 60 (9), 1295.
17. Davis, M. A., Baker, K. N., Orfe, L. H., Shah, D. H., Besser, T. E., Call, D. R. Antimicrob. Agents Chemother. 2010, 54 (6), 2666.
18. Zeng, K. J., Doi, Y, Patil, S., Huang, X., Tian, G. B. Antimicrob. Agents Chemother. 2016, 60 (6), 3862.
19. Zunk, M., Kiefel, M. J. RSC Adv. 2014, 4 (7), 3413.
20. Macauley, M. S., Crocker, P. R., Paulson, J. C. Nat. Rev. Immunol. 2014, 14 (10), 653.
21. Stephenson, H. N., Mills, D. C., Jones, H., Milioris, E., Copland, A., Dorrell, N., Wren, B. W., Crocker, P. R., Escors, D., Bajaj-Elliott, M. J. Infect. Dis. 2014, 210 (9), 1487.
22. Kenyon, J. J., Marzaioli, A. M., De Castro, C., Hall, R. M. Glycobiology 2015, 25 (6), 644.
23. Kenyon, J. J., Kasimova, A. A., Notaro, A., Arbatsky, N. P., Speciale, I., Shashkov, A. S., De Castro, C., Hall, R. M., Knirel, Y. A. Carbohydr Res 2017, 452, 149.
24. Chan, H. L., Lyu, L., Aw, J., Zhang, W., Li, J., Yang, H. H., Hayashi, H., Chiba, S., Xing, B. ACS Chem Biol 2018, 13 (7), 1890.
25. Liu, H. W., Li, K., Hu, X. X., Zhu, L., Rong, Q., Liu, Y, Zhang, X. B., Hasserodt, J., Qu, F. L., Tan, W. Angew Chem Int Ed Engl 2017, 56 (39), 11788.
26. Chyan, W., Raines, R. T. ACS Chem Biol 2018, 13 (7), 1810.
27. Liu, H. W., Chen, L., Xu, C., Li, Z., Zhang, H., Zhang, X. B., Tan, W. Chem Soc Rev 2018, 47 (18), 7140.
28. Babb, R., Pirofski, L. A., Virulence 2017, 8 (7), 1055-1058.
29. Lee, I. M., Tu, I. F., Yang, F. L., Ko, T. P., Liao, J. H., Lin, N. T., Wu, C. Y., Ren, C. T., Wang, A. H., Chang, C. M., Huang, K. F., Wu, S. H., Sci Rep 2017, 7, 42711.
30. Lee, I. M., Yang, F. L., Chen, T. L., Liao, K. S., Ren, C. T., Lin, N. T., Chang, Y. P., Wu, C. Y., Wu, S. H., J Am Chem Soc 2018, 140 (28), 8639-8643.
31. McAtee, C. P., Hoffman, P. S., Berg, D. E., Proteomics 2001, 1 (4), 516-521.
32. Medzihradszky, K. F. Methods in Enzymology 2005, 405, 50-65.
33. Suzuki, N., Quesenberry, M. S., Wang, J. K., Lee, R. T., Kobayashi, K., Lee, Y. C. Anal. Biochem. 1997, 247(2), 412-146.
34. Ren, Y, Ren, Y, Zhou, Z., Guo, X., Li, Y, Feng, L., Wang, L., J Bacteriol 2010, 192 (9), 2463-4.
35. Liu, W. Y, Wong, C. F., Chung, K. M., Jiang, J. W., Leung, F. C., PLoS One 2013, 8 (9), e74487.
36. Schoenhofen, I. C.; McNally, D. J.; Brisson, J. R.; Logan, S. M., Glycobiology 2006, 16 (9), 8C-14C.

What is claimed is:

1. A fusion protein, comprising:
a phage tail-spike protein ΦAB6TSP; and
a signal indicator.

2. The fusion protein of claim 1, wherein the signal indicator comprises luminescent molecule, chemiluminescent molecule, fluorescent dyes, fluorescence quenchers, apatite, colored molecules, radioactive isotope, scintillators, biotin, avidin, streptavidin, protein A, protein G, antibodies or fragments thereof, poly-histidine, flag tags, myc tags, HA tags, or enzymes.

3. The fusion protein of claim 2, wherein the signal indicator is DyLight-650.

4. The fusion protein of claim 3, wherein the DyLight-650 is labeled on K261, K307, K432, L476 and/or K547 of the phage tail-spike protein ΦAB6TSP.

5. The fusion protein of claim 1, wherein the phage tail-spike protein ΦAB6TSP comprises a catalytic residue mutation.

6. The fusion protein of claim 5, wherein the catalytic residue mutation comprises E447Q mutation.

7. The fusion protein of claim 6, wherein the phage tail-spike protein ΦAB6TSP comprising a sequence having at least 90% identity to SEQ ID NO: 2 or SEQ ID NO: 4.

8. A method of detecting bacteria having pseudaminic acid (Pse), comprising steps of:
contacting a sample with a phage tail-spike protein ΦAB6TSP; and
detecting a signal from the sample.

9. The method of claim 8, wherein the bacteria comprise Acinetobacter baumannii, Helicobacter pylori, Enterobacter cloacae, or Campylobacter jejuni.

10. The method of claim 8, further comprising a step of dyeing the sample prior to the contacting step.

11. The method of claim 10, further comprising a step of immobilizing the phage tail-spike protein ΦAB6TSP on a substrate.

12. The method of claim 8, wherein the phage tail-spike protein ΦAB6TSP is fused with a signal indicator.

13. The method of claim 12, wherein the signal indicator comprises luminescent molecule, chemiluminescent molecule, fluorescent dyes, fluorescence quenchers, apatite, colored molecules, radioactive isotope, scintillators, biotin, avidin, streptavidin, protein A, protein G, antibodies or fragments thereof, poly-histidine, flag tags, myc tags, HA tags, or enzymes.

14. The method of claim 13, wherein the signal indicator is DyLight-650.

15. The method of claim 14, wherein the DyLight-650 is labeled on K261, K307, K432, L476 and/or K547 of the phage tail-spike protein ΦAB6TSP.

16. The method of claim 8, wherein the phage tail-spike protein ΦAB6TSP comprises a catalytic residue mutation.

17. The method of claim 16, wherein the catalytic residue mutation comprises E447Q mutation.

18. The method of claim 17, wherein the phage tail-spike protein ΦAB6TSP comprising a sequence having at least 90% identity to SEQ ID NO: 2 or SEQ ID NO: 4.


SEQUENCE LISTING (4 sequences)

SEQ ID NO: 1 - DNA, 1695 bp, Artificial Sequence
  I-AB6TSP (WT) nucleic acid sequence
agtgaagctgctcaagaggctgctaatgcagcagaagtagctgcttcacaaacacagtactacttaaagtacttcaaccctgaaatagtgtaccctaaaaatgcgcgcattatgcttgataatggcgatattgttcgctctaccgttgtgaataatacatccaatccgaatgttgatatgactggatgggtaaaggttagttcggttagtcagatttttgatgaaacttacaatatcacccaatcagtaattaatggaaatctaattactgttgataattttggcgcaaagggtgatggtgttacagatgatagcgcagcttttcaagcatattgtgatagtgctttaactggacaaaatctatatcttggggcaaaaggtcgctacatcctcaagaatcaagtagacctaaaaggtaaaggtttggttggcaacgggtgcggaaaggttagtgagttctactacaacttaggttgtattgacgttgatggctcaagccctgatcttcaaggtaaaacggcattcattaattgtggtcctacaatccaaaatcttactgcaagatgctctaatggagcagggaaacaggtctcatttattgaaatagacggctatctagccaatattgaccacataacgcttattaatttctacaatcagattgttgttaaacaagcacttgttggttttaacttcacaaacgcttggttgtattactcacaaaatgctggaatttattgtgaagacccattgaaccgagtcagcacaactggaacatttcacaacatttatttccaattaggtgacggtcatgcaatgattttcgatagggatgtccatgggtgtgatttcgataatattatattcgaatctatgaatggaggtatcaaggctcgaacagtagcacattgcgggtttggtaaattctggtgtcagaacttaaagacagctacatccaaagattggctagaggtcactggtgctaatagctgttatgggaatagctttactggctatgttaaattattaggcgggtggacgtcgaaaacatcaccaacgctagactctttgccaacaaataactatggtggtgtatcggttagtgcagagggcatctctattgttaatgctggcaataaagcaaaaatgcttatgctgccaagtggttttaagactggcaatgcaacgattgatgaaacgcacattagctcatctaccgtaacacccttagttaagagacgtgttattggtgcagatagctctggagcacaataccttgcatcagacacctatacaaagctttctcgcaagtggggaacatataaccatggatcaaataatgctggggcgttttatgcaccaatgatgcttacttatgatcagtcatttagcacaccacaaaacaataatggttggaaaatcgttaaagaatctactggcgtttatcgggttgaacgtgtttctgggaatacttcggtaatcacaaatggacatattgttgttggctcaccactaatgggttctcgtctgggtactggaacaggtgctacgcatggaattcaaatgattgagacctatgcaggttcgtggacttcttacacagaagcggctggttttaaagtgttttggcgcgattctagcaatgccctagtcgatcctcatagatttacagtcgcatttacagcaacgagttaa

SEQ ID NO: 2 - protein, 564 aa, Artificial Sequence
  I-AB6TSP (WT) amino acid sequence
  VARIANT (1)..(8): Deletion in Trun-I-?AB6TSP (Truncation)
  DOMAIN (181)..(420): Funactional domain
  MUTAGEN (312)..(312): Catalytic residue mutation from Glu to Gln
SEAAQEAANAAEVAASQTQYYLKYFNPEIVYPKNARIMLDNGDIVRSTVVNNTSNPNVDMTGWVKVSSVSQIFDETYNITQSVINGNLITVDNFGAKGDGVTDDSAAFQAYCDSALTGQNLYLGAKGRYILKNQVDLKGKGLVGNGCGKVSEFYYNLGCIDVDGSSPDLQGKTAFINCGPTIQNLTARCSNGAGKQVSFIEIDGYLANIDHITLINFYNQIVVKQALVGFNFTNAWLYYSQNAGIYCEDPLNRVSTTGTFHNIYFQLGDGHAMIFDRDVHGCDFDNIIFESMNGGIKARTVAHCGFGKFWCQNLKTATSKDWLEVTGANSCYGNSFTGYVKLLGGWTSKTSPTLDSLPTNNYGGVSVSAEGISIVNAGNKAKMLMLPSGFKTGNATIDETHISSSTVTPLVKRRVIGADSSGAQYLASDTYTKLSRKWGTYNHGSNNAGAFYAPMMLTYDQSFSTPQNNNGWKIVKESTGVYRVERVSGNTSVITNGHIVVGSPLMGSRLGTGTGATHGIQMIETYAGSWTSYTEAAGFKVFWRDSSNALVDPHRFTVAFTATS

SEQ ID NO: 3 - DNA, 1671 bp, Artificial Sequence
  Trun-I-AB6TSP (Truncation) nucleic acid sequence
aatgcagcagaagtagctgcttcacaaacacagtactacttaaagtacttcaaccctgaaatagtgtaccctaaaaatgcgcgcattatgcttgataatggcgatattgttcgctctaccgttgtgaataatacatccaatccgaatgttgatatgactggatgggtaaaggttagttcggttagtcagatttttgatgaaacttacaatatcacccaatcagtaattaatggaaatctaattactgttgataattttggcgcaaagggtgatggtgttacagatgatagcgcagcttttcaagcatattgtgatagtgctttaactggacaaaatctatatcttggggcaaaaggtcgctacatcctcaagaatcaagtagacctaaaaggtaaaggtttggttggcaacgggtgcggaaaggttagtgagttctactacaacttaggttgtattgacgttgatggctcaagccctgatcttcaaggtaaaacggcattcattaattgtggtcctacaatccaaaatcttactgcaagatgctctaatggagcagggaaacaggtctcatttattgaaatagacggctatctagccaatattgaccacataacgcttattaatttctacaatcagattgttgttaaacaagcacttgttggttttaacttcacaaacgcttggttgtattactcacaaaatgctggaatttattgtgaagacccattgaaccgagtcagcacaactggaacatttcacaacatttatttccaattaggtgacggtcatgcaatgattttcgatagggatgtccatgggtgtgatttcgataatattatattcgaatctatgaatggaggtatcaaggctcgaacagtagcacattgcgggtttggtaaattctggtgtcagaacttaaagacagctacatccaaagattggctagaggtcactggtgctaatagctgttatgggaatagctttactggctatgttaaattattaggcgggtggacgtcgaaaacatcaccaacgctagactctttgccaacaaataactatggtggtgtatcggttagtgcagagggcatctctattgttaatgctggcaataaagcaaaaatgcttatgctgccaagtggttttaagactggcaatgcaacgattgatgaaacgcacattagctcatctaccgtaacacccttagttaagagacgtgttattggtgcagatagctctggagcacaataccttgcatcagacacctatacaaagctttctcgcaagtggggaacatataaccatggatcaaataatgctggggcgttttatgcaccaatgatgcttacttatgatcagtcatttagcacaccacaaaacaataatggttggaaaatcgttaaagaatctactggcgtttatcgggttgaacgtgtttctgggaatacttcggtaatcacaaatggacatattgttgttggctcaccactaatgggttctcgtctgggtactggaacaggtgctacgcatggaattcaaatgattgagacctatgcaggttcgtggacttcttacacagaagcggctggttttaaagtgttttggcgcgattctagcaatgccctagtcgatcctcatagatttacagtcgcatttacagcaacgagttaa

SEQ ID NO: 4 - protein, 556 aa, Artificial Sequence
  Trun-I-AB6TSP (Truncation) amino acid sequence
  DOMAIN (173)..(412): Funactional domain
  MUTAGEN (304)..(304): Catalytic residue mutation from Glu to Gln
NAAEVAASQTQYYLKYFNPEIVYPKNARIMLDNGDIVRSTVVNNTSNPNVDMTGWVKVSSVSQIFDETYNITQSVINGNLITVDNFGAKGDGVTDDSAAFQAYCDSALTGQNLYLGAKGRYILKNQVDLKGKGLVGNGCGKVSEFYYNLGCIDVDGSSPDLQGKTAFINCGPTIQNLTARCSNGAGKQVSFIEIDGYLANIDHITLINFYNQIVVKQALVGFNFTNAWLYYSQNAGIYCEDPLNRVSTTGTFHNIYFQLGDGHAMIFDRDVHGCDFDNIIFESMNGGIKARTVAHCGFGKFWCQNLKTATSKDWLEVTGANSCYGNSFTGYVKLLGGWTSKTSPTLDSLPTNNYGGVSVSAEGISIVNAGNKAKMLMLPSGFKTGNATIDETHISSSTVTPLVKRRVIGADSSGAQYLASDTYTKLSRKWGTYNHGSNNAGAFYAPMMLTYDQSFSTPQNNNGWKIVKESTGVYRVERVSGNTSVITNGHIVVGSPLMGSRLGTGTGATHGIQMIETYAGSWTSYTEAAGFKVFWRDSSNALVDPHRFTVAFTATS